(19) United States
(12) Patent Application Publication
(10) Pub. No.: US 2019/0231883 A1
(43) Pub. Date: Aug. 1, 2019

(54) SPIDER SILK PROTEIN DRUG COMPOSITIONS AND DELIVERY

(71) Applicant: Utah State University, Logan, UT (US)

(72) Inventors: Randolph V. Lewis, Nibley, UT (US); Justin A. Jones, Nibley, UT (US); Thomas Harris, North Logan, UT (US); Deven Smuin, North Logan, UT (US)

(21) Appl. No.: 16/265,663

(22) Filed: Feb. 1, 2019

Related U.S. Application Data
(60) Provisional application No. 62/625,203, filed on Feb. 1, 2018.

(51) Int. Cl. A61K 47/42; A61K 9/00; A61K 9/06; A61K 47/12; A61K 47/26

(52) U.S. Cl. CPC A61K 47/42; A61K 9/0063; A61K 47/26; A61K 47/12; A61K 9/06

(57) ABSTRACT

The present disclosure relates to drug delivery compositions that include recombinant spider silk and a medicinal agent and methods for preparing such materials and delivery medicinal agents. The disclosure also relates to compositions and methods of treating periodontal disease. A drug delivery composition can include a spider silk protein and a medicinal agent. The drug delivery composition can be in the form of a fiber, a solution, a gel, a hydrogel, a solid chip, a film, an adhesive, or a coating.

### BACKGROUND OF THE INVENTION

The present disclosure relates to drug delivery compositions that include recombinant spider silk and one or more medicinal agent and methods for preparing such materials as well as techniques for delivering medicinal agents. The disclosure also relates to methods of treating diseases, including periodontal disease.

Spider silks and other natural silks are proteinaceous fibers composed largely of non-essential amino acids. Orb-web spinning spiders have as many as seven sets of highly specialized glands that produce up to seven different types of silk. Each silk protein has a different amino acid composition, mechanical property, and function. The physical properties of a silk fiber are influenced by the amino acid sequence, spinning mechanism, and environmental conditions in which they are produced.

Dragline spider silk is among the strongest known biomaterials. It is the silk used for the framework of a spider web and used to catch the spider if it falls. For example, the dragline silk of A. diadematus demonstrates high tensile strength (1.9 GPa; ˜15 gpd) approximately equivalent to that of steel (1.3 GPa) and synthetic fibers such as aramid fibers (e.g., Kevlar™). Dragline silk is made of two proteins, Major Ampullate Spider Proteins 1 and 2 (MaSp1 and MaSp2). MaSp1 is responsible for the strength of dragline silk, while the MaSp2 is responsible for the elastic characteristics.

The physical properties of dragline silk balance stiffness and strength, both in extension and compression, imparting the ability to dissipate kinetic energy without structural failure. Due to their desirable mechanical properties, proteinaceous fibers and silks may be desirable for new biomaterials, drug delivery, tendon and ligament repair, as well as athletic gear, military applications, airbags, and tire cords among others.

Periodontal disease is an inflammatory disease that affects both soft and hard structures that support the teeth. In the more severe form of periodontal disease called periodontitis, the gums pull away from the tooth and supporting gum tissues are destroyed. Periodontitis is a chronic inflammatory disease characterized by the destruction of the periodontium due to an excessive and sustained host response to a multi-microbial insult. It affects around 47.2 million adults in the United States, and it is the lead cause of edentulism in the developed world.

Bone can be lost, and the teeth may loosen or eventually fall out. According to recent findings from the Centers for Disease Control and Prevention (CDC), half of Americans aged 30 or older have periodontitis. Reducing pocket depth and eliminating existing bacteria are important to prevent the progression of periodontal disease. Deeper pockets are more difficult for patients and dental care professionals to clean. In periodontal treatment antimicrobial chips, spheres, or gels are placed into the periodontal pocket in order to eliminate bacteria allowing the supporting gum tissue to reattach to the tooth.

The predominant paradigm for the etiology of periodontitis is the presence of a biofilm composed by what is known as the red complex: a combination of microbes including Porphyromonas gingivalis, Treponema denticola, and Tanerella forsythia. P. gingivalis was a widely accepted model for periodontal inflammation as it is easily cultured and causes inflammatory bone loss. Currently, the polymicrobial synergy and dysbiosis model (PSD) is the mainstream mechanism in the etiology of periodontitis. The PSD model compares the combination of several bacterial species in periodontal disease with their relative abundance in oral health. New sequencing techniques permitted the identification of diverse microbial communities involved in periodontitis. In a susceptible host, keystone pathogens such as P. gingivalis initiate a breakdown in homeostasis while existing commensals become proinflammatory pathobionts, which cause a dysbiotic state and promote periodontal disease.

Bacteria is essential for periodontitis to occur, however, the severity, pattern, and progression of the disease is not solely determined by the microbial burden; it is also a function of an overwhelming host inflammatory response. The response can vary even in two individuals with similar periodontopathogenic profiles. Initially, a pathogen such as P. gingivalis interacts with Toll-like receptors 2 and 4 (TLR2 and TLR4) from local cells, exploiting the TLR2/TLR4 crosstalk with the complement system (C5a) to hijack normal defense responses and chemotaxis of defense cells. Meanwhile, other virulence factors induce the production of inflammatory cytokines (interleukins, tumor necrosis factor-α), prostanoids and proteolytic enzymes, mainly matrix metalloproteinases (MMPs) that are the main causes of gingival damage.

The current status of periodontitis treatment is based in the mechanical debridement of biofilm (scaling and root planning), systemic or localized antibiotic therapy and even antimicrobial photodynamic therapy. Surgical procedures such as gingivectomy and flap debridement are used with less frequency and often accompanied by antimicrobial therapy. The sole focus of these approaches is to control the microbial invasion or repair tissue; they do not address feedback from the host response that perpetuates the disease. Although both non-surgical and surgical approaches can be effective in controlling periodontal damage, they require strict maintenance regimes and do not prevent disease in other sites.

Current treatments for periodontitis are ineffective. Arestin studies demonstrate ineffectiveness with less than 25% success rates. PerioChip also requires multiple applications/treatments with limited success. As a response to the limitations of the traditional therapies, new agents have been used in preclinical and clinical studies, namely host-modulatory agents, including anti-proteinase agents, anti-inflammatory agents and anti-resorptive agents. New therapeutic approaches should focus on mediating the inflammatory process, as opposed to focusing solely on the microbial insult. Effective control of the immune response will slow the disease progression, improve clinical outcomes and even prevent future sites of active periodontitis.

### SUMMARY OF THE INVENTION

In one aspect, a drug delivery composition is disclosed. The composition includes a spider silk protein selected from MaSp1, MaSp2, MiSp1, MiSp2, tubuliform, flagelliform, piriform, aciniform, aggregate, and any combination thereof; a medicinal agent; and optionally a carrying agent. In some embodiments, the carrying agent is water. In some embodiments, the medicinal agent is selected from an antibiotic, an anti-inflammatory agent, a growth factor, an analgesic, a bioactive, and any combination thereof.

In some embodiments, the drug delivery composition is in a form selected from: a fiber, a solution, a gel, a hydrogel, a solid chip, a film, an adhesive, and a coating. In some embodiments, the form is a fiber. In some embodiments, the form is a solution. In some embodiments, the form is a gel. In some embodiments, the form is a hydrogel. In some embodiments, the form is a solid chip. In some embodiments, the form is a film. In some embodiments, the form is an adhesive. In some embodiments, the form is a coating.

In some embodiments, the drug delivery composition also includes a cross-linking agent.

In one aspect, a method of delivering a medicinal agent is disclosed. The method includes administering to a subject the drug delivery compositions disclosed herein, wherein when the drug delivery composition is administered in a fluid or gel form, the composition fills voids on a substrate surface.

In some embodiments, the drug delivery composition is administered orally or dermally. In some embodiments, the drug delivery composition is administered sub-gingivally. In some embodiments, the method includes curing the drug delivery composition before or after the administration step. In some embodiments, the method includes contacting a target area with the medicinal agent.

In one aspect, a method of preparing a drug delivery composition is disclosed. The method includes solubilizing a spider silk protein selected from MaSp1, MaSp2, MiSp1, MiSp2, tubuliform, flagelliform, piriform, aciniform, aggregate, and any combination thereof in an aqueous solution; and adding a medicinal agent to the aqueous solution.

In some embodiments, the method also includes adding the drug delivery composition to a mold and allowing the drug delivery composition to solidify forming a solid chip in the form of the mold.

In one aspect, a method of treating periodontal disease is disclosed. The method includes administering to a subject the drug delivery composition prepared according to the any of the methods of disclosed herein.

In one aspect, an adhesive is disclosed which includes a drug delivery composition disclosed herein.

In one aspect, a coating on a medical device (coated medical device) is disclosed. The medical device includes the drug delivery composition disclosed herein.

### BRIEF DESCRIPTION OF THE DRAWINGS

FIG. 1 shows representative release profiles of PerioChips and rSSps chips. (A) Release curve for conventional PerioChip device. (B) Release curve for 12.5% (w/v) 80:20 (M4:M5) spider silk chip. (C) Release profile for 6.25% (w/v) 80:20 (M4:M5) spider silk chip. (D) Release profile for 6.25% (w/v) M4 spider silk chip.

FIG. 2 shows zone of inhibition studied for spider silk chips. Top left chip: control (no chlorhexidine). (A) Growth after 48 hours with zones of inhibition present. (B) One week after initial plating: a definitive zone is still observed. Unplanned mold contaminants were present on the control but inhibited by functionalized spider silk chips.

FIG. 3 shows daily zone inhibition measurements for both PerioChip and spider silk chips.

### DETAILED DESCRIPTION

The present disclosure covers methods, compositions, and reagents for making medical devices with coatings or adhesives from synthetic spider silk protein compositions. The synthetic spider silk proteins are sometimes referred to as of regenerated spider silk proteins (rSSp) or recombinant spider silk proteins.

Spider silks proteins and materials are biocompatible, biodegradable, tunable, and exhibit adhesive abilities to multiple surfaces (enamel and sub gingiva). The spider silk proteins can be processed and tuned to produce a diversity of products such as solid chips, gels, pastes, or solutions.

The drug release profile from drug delivery compositions can be tuned using different silk types, spider silk protein concentration, and preparation procedures.

The combination of spider silk deliver vehicles with multi-targeting medications offers a radical change in the thoughts and approaches to treating diseases, including periodontitis. By leveraging the properties of spider silks, whether those are toughness, biocompatibility, or biodegradability, and applying them to a biomedical material will guarantee refined performance and treatment. The ability to be worked into multiple forms, which also have further tunability, will provide new treatment platforms that will be able to better control release and elution rates. Furthermore, spider silks are biocompatible, and the risk of the delivery vehicle inducing an immune response is greatly decreased. Most current treatment methods also only focus on the microbial infection with antibiotic. The drug delivery composition and methods disclosed herein can target both the microbial and immune response and use combination therapies of antiseptics and anti-inflammatories.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

As used herein, the phrases “dope solution” or “spin dope” means any liquid mixture that contains silk protein and is amenable to extrusion for the formation of a biofilament or film casting. Dope solutions may also contain, in addition to protein monomers, higher order aggregates including, for example, dimers, trimers, and tetramers. Normally, dope solutions are aqueous solutions of between pH 4.0 and 12.0 and having less than 40% organics or chaotropic agents (w/v). In some embodiments, the dope solutions do not contain any organic solvents or chaotropic agents, yet may include additives to enhance preservation, stability, or workability of the solution. Dope solutions may be made by purifying and concentrating a biological fluid from a transgenic organism that expresses a recombinant silk protein. Suitable biological fluids include, for example, cell culture media, milk, urine, or blood from a transgenic mammal, cultured bacteria, and exudates or extracts from transgenic plants.

As used herein, the term “filament” means a fiber of indefinite length, ranging from microscopic length to lengths of a mile or greater. Silk is a natural filament, while nylon and polyester are synthetic filaments. A “blended filament” or “blended fiber” means a fiber that includes a silk or natural component and a synthetic component such as nylon or polyurethane for example.

As used herein, the term “toughness” refers to the energy needed to break the fiber or filament. This is the area under the stress-strain curve, sometimes referred to as “energy to break” or work to rupture.

As used herein, the term “elasticity” refers to the property of a body which tends to recover its original size and shape after deformation. Plasticity, deformation without recovery, is the opposite of elasticity. On a molecular configuration of the textile fiber, recoverable or elastic deformation is possible by stretching (reorientation) of inter-atomic and inter-molecular structural bonds. Conversely, breaking and re-forming of intermolecular bonds into new stabilized positions causes non-recoverable or plastic deformations.

As used herein, the term “fineness” means the mean diameter of a fiber which is usually expressed in microns (micrometers).

As used herein, the term “micro fiber” means a filament having a fineness of less than 1 denier.

As used herein, the term “modulus” refers to the ratio of load to corresponding strain for a fiber, yarn, or fabric.

As used herein, the term “orientation” refers to the molecular structure of a filament or the arrangement of filaments within a thread or yarn, and describes the degree of parallelism of components relative to the main axis of the structure. A high degree of orientation in a thread or yarn is usually the result of a combing or attenuating action of the filament assemblies. Orientation in a fiber is the result of shear flow elongation of molecules.

As used herein, the term “spinning” refers to the process of making filament or fiber by extrusion of a fiber forming substance, drawing, twisting, or winding fibrous substances.

As used herein, the term “tenacity” or “tensile strength” refers to the amount of weight a filament can bear before breaking. The maximum specific stress that is developed is usually in the filament, yarn or fabric by a tensile test to break the materials.

As used herein, the term “substantially pure” is meant substantially free from other biological molecules such as other proteins, lipids, carbohydrates, and nucleic acids. Typically, a dope solution is substantially pure when at least 60%, more preferably at least 75%, even more preferably 85%, most preferably 95%, or even 99% of the protein in solution is silk protein, on a wet weight or a dry weight basis. Further, a dope solution is substantially pure when proteins account for at least 60%, more preferably at least 75%, even more preferably 85%, most preferably 95%, or even 99% by weight of the organic molecules in solution.

### Suitable Silk Proteins

A variety of silk proteins can be used in the processes described herein. They include proteins from plant and animal sources, as well as recombinant and other cell culture source such as bacterial cultures. Such proteins may include sequences conventionally known for silk proteins (see for example, U.S. Pat. No. 7,288,391, incorporated herein by reference in its entirety).

Suitable spider silk proteins may be derived from conditioned media recovered from eukaryotic cell cultures, such as mammalian cell cultures, which have been engineered to produce the desired proteins as secreted proteins. Cell lines capable of producing the subject proteins can be obtained by cDNA cloning, or by the cloning of genomic DNA, or a fragment thereof, from a desired cell. Examples of mammalian cell lines useful for the practice of the invention include, but are not limited to BHK (baby hamster kidney cells), CHO (Chinese hamster ovary cells) and MAC-T (mammary epithelial cells from cows).

The spider silk proteins may be from several recombinant sources. Examples of such proteins recombinantly expressed include those identified in U.S. patent application No. 61/707,571; Ser. No. 14/042,183; PCT/US2013/062722; 61/865,487; and 61/917,259 that are incorporated herein by reference in their entirety, including recombinantly produced major ampullate, minor ampullate, flagelliform, tubuliform, aggregate, aciniform and piriform proteins. These proteins may be any type of biofilament proteins such as those produced by a variety of arachnids including, for example, Nephila clavipes, Araneus ssp. and A. diadematus. Also suitable for use in the invention are proteins produced by insects such as Bombyx mori. Dragline silk produced by the major ampullate gland of Nephila clavipes occurs naturally as a mixture of at least two proteins, designated as MaSpI and MaSpII. Similarly, dragline silk produced by A. diadematus is also composed of a mixture of two proteins, designated ADF-3 and ADF-4.

The spider silk proteins may be monomeric proteins, fragments thereof, or dimers, trimers, tetramers or other multimers of a monomeric protein. The proteins are encoded by nucleic acids, which can be joined to a variety of expression control elements, including tissue-specific animal or plant promotors, enhancers, secretory signal sequences and terminators. These expression control sequences, in addition to being adaptable to the expression of a variety of gene products, afford a level of control over the timing and extent of production.

Suitable spider silk proteins may be extracted from mixtures comprising biological fluids produced by transgenic animals, such as transgenic mammals, including goats. Such animals have been genetically modified to secrete a target biofilament in, for example, their milk or urine (see for example, U.S. Pat. No. 5,907,080; WO 99/47661 and U.S. patent publication Ser. No. 20010042255, all of which are incorporated herein by reference). The biological fluids produced by the transgenic animals may be purified, clarified, and concentrated, through such techniques as, for example, tangential flow filtration, salt-induced precipitation, acid precipitation, EDTA-induced precipitation, and chromatographic techniques, including expanded bed absorption chromatography (see for example U.S. patent application Ser. No. 10/341,097, entitled Recovery of Biofilament Proteins from Biological Fluids, filed Jan. 13, 2003, incorporated herein by reference in its entirety).

The suitable spider silk proteins may originate from plant sources. Several methods are known in the art by which to engineer plant cells to produce and secrete a variety of heterologous polypeptides (see for example, Esaka et al., Phytochem. 28:2655 2658, 1989; Esaka et al., Physiologia Plantarum 92:90 96, 1994; and Esaka et al, Plant Cell Physiol. 36:441 446, 1995, and Li et al., Plant Physiol. 114:1103 1111). Transgenic plants have also been generated to produce spider silk (see for example Scheller et al., Nature Biotech. 19:573, 2001; PCT publication WO 01/94393 A2).

Exudates produced by whole plants or plant parts may be used. The plant portions can be intact and living plant structures. These plants materials may be a distinct plant structure, such as shoots, roots or leaves. Alternatively, the plant portions may be part or all of a plant organ or tissue, provided the material contains or produces the biofilament protein to be recovered.

Having been externalized by the plant or the plant portion, exudates are readily obtained by any conventional method, including intermittent or continuous bathing of the plant or plant portion (whether isolated or part of an intact plant) with fluids. Exudates can be obtained by contacting the plant or portion with an aqueous solution such as a growth medium or water. The fluid-exudate admixture may then be subjected to the purification methods of the present invention to obtain the desired biofilament protein. The proteins may be recovered directly from a collected exudate, such as a guttation fluid, or a plant or a portion thereof.

Extracts may be derived from any transgenic plant capable of producing a recombinant biofilament protein. Plant species representing different plant families, including, but not limited to, monocots such as ryegrass, alfalfa, turfgrass, eelgrass, duckweed and wilgeon grass; dicots such as tobacco, tomato, rapeseed, azolla, floating rice, water hyacinth, and any of the flowering plants may be used. Other useful plant sources include aquatic plants capable of vegetative multiplication such as Lemna, and duckweeds that grow submerged in water, such as eelgrass and wilgeon grass. Water-based cultivation methods such as hydroponics or aeroponics are useful for growing the transgenic plants of interest, especially when the silk protein is secreted from the plant's roots into the hydroponic medium from which the protein is recovered.

Spider silk proteins are designated according to the gland or organ of the spider in which they are produced. Spider silks known to exist include major ampullate (MaSp), minor ampullate (MiSp), flagelliform (Flag), tubuliform, aggregate, aciniform, and piriform spider silk proteins. Spider silk proteins derived from each organ are generally distinguishable from those derived from other synthetic organs by virtue of their physical and chemical properties. For example, major ampullate silk, or dragline silk, is extremely tough. Minor ampullate silk, used in web construction, has high tensile strength. An orb-web's capture spiral, in part composed of flagelliform silk, is elastic and can triple in length before breaking. Tubuliform silk is used in the outer layers of egg-sacs, whereas aciniform silk is involved in wrapping prey and piriform silk is laid down as the attachment disk.

Sequencing of spider silk proteins has revealed that these proteins are dominated by iterations of four simple amino acid motifs: (1) polyalanine (Alaₙ); (2) alternating glycine and alanine (GlyAla)ₙ; (3) GlyGlyXaa; and (4) GlyProGly(Xaa)ₙ, where Xaa represents a small subset of amino acids, including Ala, Tyr, Leu and Gln (for example, in the case of the GlyProGlyXaaXaa motif, GlyProGlyGInGln is the major form). Spider silk proteins may also contain spacers or linker regions comprising charged groups or other motifs, which separate the iterated peptide motifs into clusters or modules.

In some embodiments, suitable spider silk proteins that can be used include recombinantly produced MaSp1 (also known as MaSpI) and MaSp2 (also known as MaSpII) proteins; minor ampullate spider silk proteins; flagelliform silks; and spider silk proteins described in any of U.S. Pat. Nos. 5,989,894; 5,728,810; 5,756,677; 5,733,771; 5,994,099; 7,057,023; and U.S. provisional patent application No. 60/315,529 (all of which are incorporated herein by reference).

The sequences of the spider silk proteins may have amino acid inserts or terminal additions, so long as the protein retains the desired physical characteristics. Likewise, some of the amino acid sequences may be deleted from the protein so long as the protein retains the desired physical characteristics. Amino acid substitutions may also be made in the sequences, so long as the protein possesses or retains the desired physical characteristics.

Spider silk protein, for example MaSp1, was be blended with a synthetic material, for example nylon 66, to study its influence on crystallization and the mechanical properties of the produced yarns. The prepared dopes were spun into nanofiber mats and twisted into yarns. The electrospinning method was chosen as a nanofiber production method due to its versatility and simplicity. The electrospun fibers were aligned on a metallic cylinder and then twisted manually into yarns. The mechanical, thermal, and optical characterizations were then investigated.

In general, methods of preparing aqueous dopes of rSSp may include the following steps: mixing rSSp, water, and optional additives; optionally sonicating the mixture; microwaving the mixture; and optionally centrifuging the mixture to solubilize the rSSps.

rSSp and water are combined to create a doping mixture of greater than about 2% w/v (e.g. 0.02 g rSSp: 1 mL H₂O). In embodiments, the w/v does not typically exceed 50%. However, any percentage of less than 50% may be used.

Suitable rSSps include: MaSp1 (as described in U.S. Pat. Nos. 7,521,228 and 5,989,894), MaSp2 (as described in U.S. Pat. Nos. 7,521,228 and 5,989,894), MiSp1 (as described in U.S. Pat. Nos. 5,733,771 and 5,756,677), MiSp2 (as described in U.S. Pat. Nos. 5,733,771 and 5,756,677) , Flagelliform (as described in U.S. Pat. No. 5,994,099), chimeric rSSps (as described in U.S. Pat. No. 7,723,109), Piriform, aciniform, tubuliform, aggregate gland silk proteins, and AdF-3 and AdF-4 from Araneus diadematus. Each of the above referenced patents is herein incorporated by reference in its entirety.

### Dope Additives

Various optional additives may be optionally added to the mixture. Suitable additives include compositions that contribute to the solubility of the rSSp in the solution. Some additives break or weaken disulfide bonds, thereby increasing the solubility of rSSps. Other additives also serve to prevent hydrogel formation after the completion of the microwave heating step. If the solution forms a hydrogel quickly and the desired end product is not a gel, then additives capable of delaying or inhibiting such a formation may be desirable. In some embodiments, multiple additives may be added to achieve desired end products.

For example, to combat hydrogel formation, various additives may be added to the suspension of rSSp and water prior to microwaving the suspension. In some embodiments, acid, base, free amino acids, surfactants, or combinations thereof may be employed to combat hydrogel formation. For example, additions of acid (formic acid and acetic acid alone or together at 0.1% to 10% v/v), base (ammonium hydroxide at 0.1% to 10% v/v), free amino acids (L-Arginine and L-Glutamic Acid at 1 to 100 mM) as well as a variety of surfactants (Triton X-100 at 0.1% v/v) may be used. The additions of these various chemicals not only aid the solubility of rSSp when microwaved but in certain combinations also delay the solution from turning into a hydrogel long enough for the solution to be applied as a coating or adhesive.

Exemplary additives also include compositions capable of breaking or weakening disulfide bonds, such as p-mercaptoethanol or dithiothreitol may be added to reduce bonds and increase solubility. Suitable amounts of such additives may include from about 0.1 to about 5% (v/v). In embodiments where the rSSp does not contain cysteine, the use of such additives may be unnecessary. In some embodiments employing major ampullate silk proteins 1 and 2 (MaSp1 and MaSp2, respectfully), disulfide bonds (cysteine) are present in the C-terminus of the non-repetitive regions of MaSp1 and MaSp2. These proteins are described in U.S. Pat. Nos. 7,521,228 and 5,989,894, the entirety of which is herein incorporated by reference. In addition, the C-term is present in various goat-derived spider silk proteins M4, M5 and M55 proteins, which are described in U.S. Patent Application Publication No. 20010042255 A1, the entirety of which is incorporated by reference in its entirety. In some embodiments, formic acid and/or acetic acid may be included in as little as 0.3% (v/v) but even lower amounts (0.1% v/v) are possible. Additionally, it is possible to solubilize rSSp without using any additives.

Exemplary additives are set forth in Table 1 (below), where dope formulations prepared according to the methods described herein.

TABLE 1
Additives

| | | 3 | | | |
|---|---|---|---|---|---|
| | | Free | 4 | | |
| 1 | 2 | Amino | Disulfide | 5 | 6 |
| Acid | Base | Acids | Reduction | Other | Drying Agent |
| Acetic | Ammonium | Arginine | β- | Triton X-100 | Methanol |
| | Hydroxide | | mercaptoethanol | | |
| Formic | Sodium | Glutamic | Dithiothreitol | Glutaraldehyde | Ethanol |
| | Hydroxide | Acid | | | |
| Trifluoroacetic | | Histidine | | Calcium | Propanol |
| acid | | | | | |
| Other Organic | | Glycine | | Potassium | |
| Acids | | | | | |
| Propionic | | Imidazole | | Other Surfactants | |
| Acid | | | | | |
| | | Other | | Other Ions | |
| | | Free | | | |
| | | Amino | | | |
| | | Acids | | | |
| | | | | L-DOPA | |

In some embodiments, aqueous spin dopes omit additives. In some embodiments, the aqueous spin dope includes imidazole. In some embodiments, the aqueous spin dope includes propionic acid.

To formulate an aqueous solution of rSSp, additives can be chosen from any of the 6 columns or other additives described herein. For instance, one or a combination of acids can be chosen from column 1 and combined with one or combinations of free amino acids from column 3, as well as disulfide reducing compounds from column 4 and “Other” additives as required or desired by the particular protein or application. Generally, it would not be useful to include both an acid from column 1 with a base from column 2. However, a base from column 2 can be combined with additives from columns 3-4.

In some embodiments, the additive reduces the drying time of the aqueous dope after it is applied to a surface. Examples of such additives include those listed in column 6 of Table 1. Other alcohols may be used so long as they increase the rate of evaporation relative to distilled water.

In some embodiments, the aqueous dopes may be augmented with bicarbonate solution. The bicarbonate solution may be of from 0.001-1 M bicarbonate. The bicarbonate solution may be from 0.01 to 1 M bicarbonate. The bicarbonate solution may be 0.1 to 1.0 M bicarbonate. The bicarbonate may be from ammonium, alkaline, and alkaline earth bicarbonate, e.g. sodium bicarbonate, potassium bicarbonate, calcium bicarbonate. In some embodiments, the bicarbonate is from ammonium bicarbonate.

### Spin Dope Preparation

Spin dopes may be created using 10-40% weight protein/volume solvent (w/v). Spin dopes may be created using a variety of solvents and mixtures. In some embodiments, the primary solvent is 1,1,1,3,3,3-hexafluoro-2-proponal (HFIP) which may be augmented with additives such as formic acid, propionic acid, anhydrous toluene, acetic acid, and isopropanol. In some embodiments, HFIP is the predominant constituent making up between 70 and 100% of the total volume of a spin dope. In some embodiments, organic acids can also be included, using up to 15% of each, in order to make a spin dope. Examples of suitable organic acids include formic acid, acetic acid, and propionic acid. In some embodiments, water is included in HFIP dopes, up to 50% of the volume. Water alone can be used for creating the spin dope for some of the polymers and proteins.

For example, spider dragline silk is composed of two proteins major ampullate silk protein 1 (MaSp1) and major ampullate silk protein 2 (MaSp2). Naturally, Nephila clavipes uses a ratio of 80% MaSp1 and 20% MaSp2. Shortened versions of these proteins can be used, generated by genetically altered goats. For the creation of synthetic fibers, varying ratios of MaSp1-like and MaSp2-like protein can be used in spin dopes, from 0-100% of either can be used to make fibers with appreciable properties. Other components can be added to the spin dope for solvation, preservation, and to impart desirable physical characteristics.

To create the dopes, protein is placed in a glass vial. Solvents are then added, and the vials is placed on a motorized rotator and allowed to slowly mix. Formic acid dopes require approximately 12 hours to completely mix. Acetic acid dopes using 25-30% protein can take up to 3 days to completely dissolve. Once the protein is dissolved, impurities exist and can be removed by centrifugation. Microwave heating can be used to accelerate this process.

### Fiber Spinning

Electrospinning for the formation of the fibers disclosed herein can be used. In this electrostatic technique, a strong electric field is generated between a polymer solution contained in a glass syringe with a capillary tip and a metallic collection screen. When the voltage reaches a critical value, the charge overcomes the surface tension of the deformed drop of suspended polymer solution formed on the tip of the syringe, and a jet is produced. The electrically charged jet undergoes a series of electrically induced bending instabilities during passage to the collection screen that results in stretching. This stretching process is accompanied by the rapid evaporation of the solvent and results in a reduction in the diameter of the jet. The dry fibers accumulated on the surface of the collection screen form a non-woven mesh of nanometer to micrometer diameter fibers even when operating with aqueous solutions at ambient temperature and pressure. The electrospinning process can be adjusted to control fiber diameter by varying the charge density and polymer solution concentration, while the duration of electrospinning controls the thickness of the deposited mesh.

Electrospinning offers an effective approach to protein and synthetic component fiber formation that can potentially generate very thin fibers. Electrospinning silk fibers for biomedical applications is a complicated process, especially due to problems encountered with conformational transitions of silkworm fibroin during solubilization and reprocessing from aqueous solution to generate new fibers and films.

### Medicinal Agent

The medicinal agent can be anti-microbial agent, an anti-clotting agent, a therapeutic agent, an antibiotic, an anti-inflammatory agent, a growth factor, an analgesic, and any combination thereof.

Representative anti-microbial agents include those which kill microorganisms or inhibit their growth. Examples include antibacterial and antifungal agents.

Examples of antibacterial agents include: ceftobiprole, ceftaroline, clindamycin, calbavancin, daptomycin, linezolid, mupirocin, oritavancin, tedizolid, telavancin, tigecycline, and vancomycin. Other examples of antibacterial agents include: aminoglycosides, carbapenems, ceftazidime, cefepime, fluoroquinolones, piperacillin, ticarcillin. Still other examples of antibacterial agents include: amikacin, gentamicin, kanamycin, neomycin, netilmicin, tobramycin, paromomycin, streptomycin, spectinomycin. Still other examples of antibacterial agents include: geldanamycin, herbimycin, and rifaximin. Still other examples of antibacterial agents include: loracarbef. Still other examples of antibacterial agents include: ertapenem, doripenem, imipenem/cilastatin, meropenem. Still other examples of antibacterial agents include: cefadroxil, cefazolin, cefalotin, cephalexin. Still other examples of antibacterial agents include: cefaclor, cefamandole, cefoxtin, cefprozil, cefuroxime, cefixime, cefdinir, cefditoren, cefoperazone, cefotaxime, cefpdoxime, ceftazidime, ceftibuten, ceftizoxime, and ceftriaxone. Still other examples of antibacterial agents include cefpime. Still other examples of antibacterial agents include: ceftaroline fosamil and ceftobiprole. Still other examples of antibacterial agents include: teicoplanin, vancomycin, telavancin, dabavancin, and oritavancin. Still other examples of antibacterial agents include: clindamycin and lincomycin. Still other examples of antibacterial agents include daptomycin. Still other examples of antibacterial agents include: azithromycin, clarithromycin, dithromycin, erythromycin, roxithromycin, troleandomycin, telithromycin, and spiramycin. Still other examples of antibacterial agents include aztreonam. Still other examples of antibacterial agents include: furazolidone and nitrofurantoin. Still other examples of antibacterial agents include: linezolid, posizolid, radezolid, torezolid. Still other examples of antibacterial agents include: amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin. Still other examples of antibacterial agents include: bacitracin, colistin, and polymyxin B. Still other examples of antibacterial agents include: ciprofloxacin, enoxacin, gatifloxacin, levofloxacin, lomefloxacin, moxiflacacin, nalidxic acid, norflacin, ofloxacin, trovafloxacin, grepafloxacin, sparfloxacin, temafloxacin, mafenide, sulfacetamide, sulfadizazine, silver sulfadazine, sulfadimethoxine, sulfamethizole, sulfamethoxazole, sulfanilamide, sulfasalazine, sulfisoxacole, trimethoprim-sulfamethoxazole, sulfonamidochrysoidine. Still other examples of antibacterial agents include: demeclocycline, doxycycline, minocycline, oxytetracycline, tetracycline. Still other examples of antibacterial agents include: clofazimine, dapsone, capreomycin, cycloserine, ethambutol, ethionamide, isoniazid, pyrazinamide, rifampicin, rifabutin, rifapentine, streptomycin. Still other examples of antibacterial agents include: arsphenamine, chloramphenicol, fosformycin, fusidic acid, metronidazole, mupirocin, platensimycin, quinupristin/dalfopristin, thiampenicol, tigecycline, tinidazole, and trimethorprim. Still other examples of antibacterial agents include combinations of the foregoing.

In some embodiments, the medicinal agent is chlorhexidine gluconate.

In some embodiments, the antimicrobial agent is an anti-fungal agent. The anti-fungal agent may be selected from amphotericin B, candicidin, filipin, hamycin, natamycin, nystatin, and rimocidin. The anti-fungal agent may be selected from bifonazole, butoconazole, clotrimazole, econazole, fenticonazole, isoconazole, ketoconazole, luliconazole, miconazole, omoconazole, oxiconazole, sertaconazole, sulconazole, tioconazole. The anti-fungal agent may be selected from albaconazole, efinaconazole, epoxiconazole, fluconazole, isavuconazole, itraconazole, posaconazole, propiconazole, ravuconazole, terconazole, and voriconazole.

The anti-fungal agent may be selected from abafungin, amorolfin, butenafine, naftifine, terbinafine, echinocandins, anidulafungin, caspofungin, micafungin. The anti-fungal agent may be selected from benzoic acid, ciclopirox, flucytosine, griseofulvin, haloprogin, tolnaftate, undecylenic acid, crystal violet.

In some embodiments, the medicinal agent is an anti-clotting agent. In some embodiments, the anti-clotting agent is a coumarin (vitamin K antagonists) such as warfarin, acenocoumarol, phenprocoumon, atrometnin, and phenindone. In some embodiments, the anti-clotting agent is heparin. In some embodiments, the anti-clotting agent is a synthetic pentasaccharide inhibitor of factor Xa such as fondaparinux and idraparinux. In some embodiments, the anti-clotting agent is a direct factor Xa inhibitor such as rivaroxaban, apixaban, edoxaban, betrixaban, darexaban, letaxaban, and eribaxaban. In some embodiments, the anti-clotting agent is a direct thrombin inhibitor such as hirudin, lepirudin, bivalirudin, argatroban, dabigatran. In some embodiments, the anti-clotting agent is an antithrombin protein including antithrombin and recombinant antithrombin. In some embodiments, the anti-clotting agent is aspirin.

In some embodiments, the medicinal agent is a therapeutic agent. Examples of therapeutic agents include a variety of agents including those which have an intended therapeutic outcome for a patient need of a necessary treatment. In some embodiments, the therapeutic agent is a growth factor such as a protein or steroid hormone. In some embodiments, the growth factor is selected from adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF), fetal bovine somatotrophin (FBS), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), keratinocyte growth factor (KGF), migration-stimulating factor (MSF), myostatin (GDF-8), nerve growth factor (NGF) and other neurotrophins, platelet-derived growth factor (PDGF), thrombopoietin (TPO), T-cell growth factor (TCGF), transforming growth factor alpha (TGF-α), transforming growth factor beta (TGF-β), tumor necrosis factor-alpha (TNF-α), vascular endothelial growth factor (VEGF), Wnt signaling pathway, placental growth factor (PGF), IL-1—Cofactor for IL-3 and IL-6, IL-2—T-cell growth factor, IL-3, IL-4, IL-5, IL-6, IL-7, and Renalase—RNLS—Anti-apoptotic survival factor.

In some embodiments, the therapeutic agent is a cell adhesion factor. Examples of cell adhesion factors include cadherins, immunoglobulin superfamily (Ig) CAMs, integrins, selectins. In some embodiments, the cell adhesion factor is an RGD peptide.

### Coating and Adhesive Formation

Coatings may be produced by applying a dope solution onto a substrate and allowing the water and any volatile additives to evaporate.

Coatings prepared by the techniques disclosed herein can vary in their dimensions. Coating thicknesses can vary from 0.5 μm to 50 μm. In some embodiments, the coating thickness is from 1 to 25 μm. In some embodiments, the coating thickness is from 1 to 10 μm. In some embodiments, the coating thickness is from 1 to 25 μm.

Surfaces may be spray coated. The spray coatings can be applied in layers by applying a first coating followed by a period of drying. Following sufficient drying, second and subsequent coatings may be applied thereby creating layers of coatings. Optionally, between spray coatings, a different material such as medicinal agent may be added to a coating surface before another layer of recombinant spider silk solution is added. In some embodiments, the initial coating layer applied to a substrate surface is limited to a thin base layer to avoid beading or running on the surface. Subsequent layers added can be thicker.

Surfaces may also be dip coated. Dip coatings are prepared by taking the substrate or substrate surface and immersing it in an aqueous solution of solubilized recombinant spider silk. Once immersed, the substrate is removed to dry. Multiple layers of recombinant spider silk may be made to a surface by repeatedly dipping the surface or existing layer into the aqueous solution of solubilized recombinant spider silk followed by a brief drying period. In some embodiments, in between dip coatings, a different material such as a medicinal agent may be added to a coating surface before another layer of recombinant spider silk is added.

A combination of spray and dip coatings may also be applied. In one embodiment, an initial spray coated layer is applied to a substrate surface. Additional dip coatings may be applied to the initial spray coating. In such embodiments, surfaces treated with one or more spray coatings prior to dip coatings help to create an even coat and reduce beading and running. This combination of techniques also leads to better attachment for thicker coatings.

In addition, the techniques used here permit the preparation of medical devices that have coatings that facilitate hydrophobic substrates and, therefore, better biocompatibility and greater coating bond strengths. Also, adhesive strengths of the adhesives disclosed herein are superior to conventional adhesives that outperform conventional adhesion techniques depending on the substrate, silk type, and preparation method.

The solubilization process allows for coatings to be functionalized and tailored for specific purposes and functions. Functionalized coatings can include compounds such as therapeutic agents that are released on a predictable timescale from the recombinant spider silk coating. Functionally active coatings can prevent microbial growth and proliferation for short and longer periods such as a two week period. As discussed in the examples below, coatings have been functionalized with heparin, kanamycin, gentamicin, tetracycline, ampicillin, chloramphenicol, dexamethasone, azoles, and experimental antifungal compounds.

### Medical Device

In one aspect, a medical device is disclosed. The device can be any medical device contemporary for use in treating patients and animals that has a surface that would benefit from being coated or that has parts that can be adhered to one another.

The devices are prepared by solubilizing one or more recombinant spider silk proteins in an aqueous solution. A substrate surface is then coated with the solubilized recombinant spider silk proteins and medicinal agent. In embodiments where the coating is desired, the coated surface is then dried.

In embodiments where the device has two surfaces that need to adhere to one another, the method also includes providing a second object having a target surface that, when contacted with the substrate surface of a first object and solubilized, adheres with a first object. The adhesion can then be dried.

Exemplary medical devices can be made of a variety of materials, including components made of differing materials. For example, the device or component material and, therefore, the substrate surface can be made up of a cellulosic polymer, a silicone polymer, a plastic polymer, and a metal and combinations or segments of the same. The second object or component can be made of the same material or a different material.

In some embodiments, the plastic polymer is selected from polyurethane (PU), polystyrene (PS), polycarbonate (PC), polyethylene (PE), polypropylene (PP), expanded Teflon (ePTFE), rubber, and latex. In some embodiments, the silicone polymer is silicone. In some embodiments, the metal is selected from stainless steel, titanium, and aluminum. In some embodiments, the cellulosic polymer is wood.

Exemplary devices include a catheter, a splint, a bandage, a drain tube, and an implant. Orthopedic devices can also include coatings or adhesions as described herein.

In some embodiments, a syringe may contain any of the compositions or coating described herein.For example, the solution comprising the rSSp and water solutionsare sealed in a vial, then heated and pressurized. This solubilizes and sterilizes the rSSp solution. The solution can then be loaded into a syringe and allowed to turn to a stable hydrogel. That hydrogel in the syringe can then be placed into a the syringe heater where it transform back into a liquid that can be easily expelled at the time of use.

### EXAMPLES

Materials

Spider silk proteins were obtained through the purification of milk from transgenic goats expressing the spider silk proteins MaSp1 (M4) and MaSp2 (M5). Tested in this experiment were preparations of 25% (w/v), 12.5% (w/v), and 6.25% (w/v) concentrations of spider silk protein with ratios of 80:20 M4:M5 and 100% M4. The rSSps are first solubilized in deionized water with mild heat (>130° C.) and pressure. The heat is best applied through microwave irradiation. These conditions mildly denature the proteins and force them into the aqueous solution. Once the rSSps are in solution chlorhexidine gluconate (CHG) was added to the solubilized dope in a 1:1 mixture. This addition brought the final concentrations of silk to 12.5% (w/v), 6.25% (w/v), and 3.125% (w/v). This solution was then used to form the chips by pipetting 100 μL of the solution onto a polydimethylsiloxane (PDMS) mold and allowed to form and cure overnight.

To study gelation behavior, the gel was reheated in an oven or a syringe heater to roughly 150° C. for 10 minutes. This process resolubilizes the gelled dope with the added CHG component. The resolubilized dope is allowed to cool to body temperature, transferred to a syringe, and was extruded at two minute intervals to observe gelation and other characteristics.

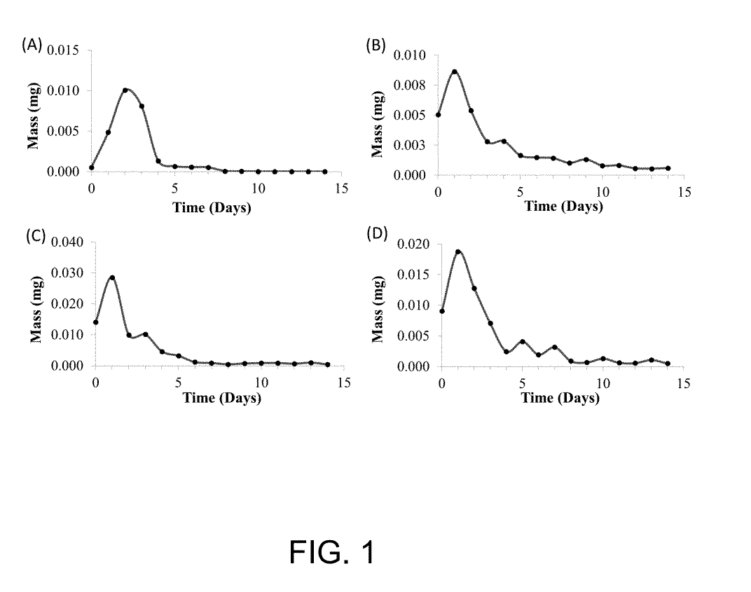

These periodontal chips were then placed into 5 mL of phosphate buffered saline (PBS) and allowed to release the medication. In order to track the amount of CHG released, samples were taken each day, and new PBS was added. This process was repeated for fourteen days. In order to test the amount of CHG released we used reverse phase ultra-high pressure liquid chromatography (RP-UPLC). Using known standards, the amount of CHG released could be calculated based upon the corresponding absorbance amount and retention time. All of the samples, both spider silk and PerioChips were analyzed with this method. FIG. 1 shows the release profiles of the various chips tested.

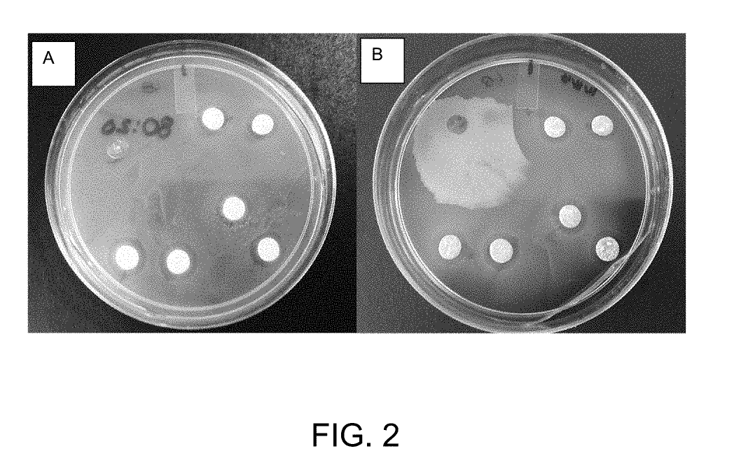

Antimicrobial activity was tested and observed during this project. The chips were placed on a plate of E. coli after spreading the cells and placed in a 37° C. incubator and observed over fourteen days. FIG. 2 shows that the chips inhibit microbial growth.

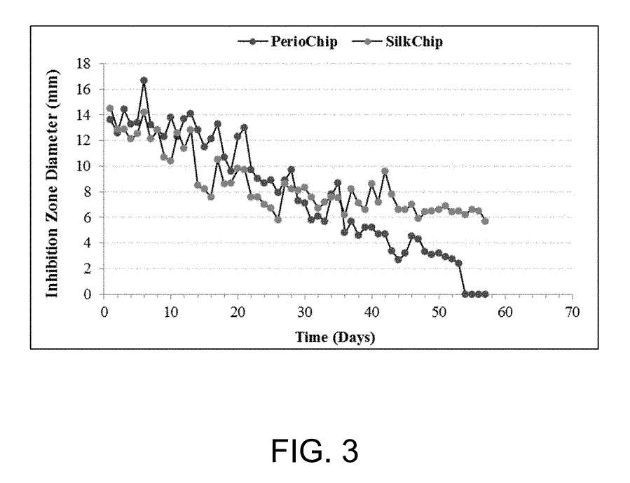

FIG. 3 shows a comparison of E. coli growth inhibition between PerioChip and a silk chip over a period of 57 days. In this experiment, each chip was placed on an agar plate that had been spread with E. coli. Each day the zone of inhibition diameter was measured and the chips were then placed on a new agar plate with a freshly spread lawn of bacteria.

Other chips were made using the process described above but with different mold sizes and drug doping amounts:
25% (w/v) dope+50/50 CHG to dope=12.5% (w/v) dope/CH solution
30 μL onto PDMS mold (4 mm×5 mm)=3.201 mg CH per chip

The amount of drug in the dope can range from about 60:40 CH:dope to about 40:60.

Another chip was made that included propionic acid to alter gelling time.

A composition of 12.5% (w/v) of spider protein and CH and 6.25% (w/v) propionic acid was made and it gelled within 10-20 minutes.

A composition of 16.5% (w/v) spider protein and CH and 6.25% (w/v) propionic acid gelled within 5 minutes.

A composition of 16.5% (w/v) spider protein and CH and 3.25% propionic acid gelled in 5-10 minutes when placed on an agar plate. This gel was more firm than 12.5% (w/v) gel.

A composition of 12.5% (w/v) spider protein and CH and 3.25% propionic acid gelled in 10-15 minutes on an agar plate.

Chips containing 12.5% (w/v) spider protein with 200 μM 1,2,3,4,6-penta-O-galloyl-β-D-glucose (PGG) were made. They were very clear and 290 μm thick.

Chips containing 6.25% (w/v) spider protein final concentration with 1:1 rSSps:CHG were roughly 265 μm thick

Chips containing 4.5% (w/v) spider protein, 3.25% (w/v) glutaraldehyde cross-linker with 1:1 weight ratio of rSSps:CHG. These chips were 200 μm thick.

A composition of 12% (w/v) of M4 with 1:1 rSSps:CHG & 0.5% propionic acid gelled in 3 minutes in great working ability with very strong set up, even better than 80:20. After 6 minutes, the gel completely set up. The pH was about 4.

A composition of 12% (w/v) of 80:20 M4:M5 with 1:1 weight ratio of rSSps:chlorhexdine and 0.5% propionic acid gelled in 6-8 minutes. After 11 minutes, the composition was not workable. After this experiment, we determined that 100% M4 has demonstrated the best working time as well as structure and strength. We used agar plates to pore onto as well as alcohol and water baths. Again M4 gave the best results.

#2—12% 6.25% acetic acid 1:1 Chlor
Gelled within 4 minutes and was workable until then. (Results 5-11)

#1—12% 0.5% acetic acid 1:1 Chlor
Gelled within 3 minutes but was not that workable during that time frame. It was very hard to get into solution and it was cloudier. (Results 1-4)

#3—16% 6.25% acetic acid 1:1 Chlor
Very hard to get into solution and gelled extremely fast. 1-2 minutes (Results 1-5 Hydrogel 2)

#4—12% 0.5% acetic acid NO Chlor
Very workable and very strong. Clear gel. (Results 6-10 Hydrogel 2)

12% M4 6.25% acetic acid
Gelation within 4 minutes and best working time within 2 minutes. Opened at 155° C.

12% M4 3:1 Acid to CHG 6.25% acetic acid
Dope gelled too fast to try.

8% M4 1:1 6.25% acetic acid
Open at 130° C., set very fast and it was hard to extrude maybe because of changed syringe tips

8% M4 3:1 Acid to CHG 6.25% acetic acid
Opened at 150° C. Workable all the way to 10 minutes; it did not seem as hard as the 3:1.

8% M4 1:1 3.125% acetic acid
Opened at 130° C. Perfect gelation at 5 minutes seems very strong. Results

8% M4 3:1 Acid to CHG 3.125% acetic acid
Opened at 130° C. Best gelation at 10 minutes but was still liquid at 30 minutes.

8% M4 w/ 1:1 CHG:H₂O formulation: gelation started in 30 minutes after the test began and was workable for up to 50 minutes. Very good consistency and color.

12% M4 w/ 1:1 CHG:H₂O formulation: gelation started in just 4 minutes after test began and only lasted until 10 minutes. Very short window of gelation period/workability. Good color and consistency.

8% M4 w/ 1:3 CHG:H₂O+10% (v/v) 0.5 M OH formulation: gelation started in just 2 minutes after first extrusion. Although it was gelled, it stayed the same consistency until 20 minutes, thus allowing for more gel to be extruded.

8% M4 w/ 1:3 CHG:H₂O dope+1% (v/v) 0.5 M OH formulation: gelation started at 10 minutes after extrusion and lasted until about 33 minutes. This gel was very white and seemed to be more aerosolized.

8% M4 w/ 1:3 CHG:H₂O dope+10% (v/v) propionic acid formulation: this combination had too much acid present resulting in almost immediate gelation and no testing.

8% M4 w/ 1:3 CHG:H₂O dope+1% (v/v) propionic acid formulation: the gelation period was about 120 minutes resulting in a clear and consistent gel.

8% M4 w/ 1:3 CHG:H₂O dope+2.5% (v/v) propionic acid formulation: started gelling in 24 minutes. The gelation period lasted until 50 minutes with roughly the same consistency resulting in a clear gel that associated with itself well and exhibited adhesive abilities with other materials/items.

8% M4 w/ 1:3 CHG:H₂O dope+5% (v/v) propionic acid formulation: started gelling at 12 minutes, and the gelation period lasted until 25 minutes with the same consistency.

In summary, the higher concentrations of spider silk proteins in the gels create firmer more robust gel, which also solidifies much faster making manipulation and extrusion difficult/unlikely. Higher concentrations of CHG prolong the gelation period and reduce the final structural consistency of the gels. Generally the higher concentration of either acid or base the faster the gel will solidify. However, acidic formulations tend to produce more robust products.

Four flasks were seeded with human gingival fibroblast (HGF-1) cells. A control flask (no chips) was prepared. Next, three sterile chips from each group were placed in each of the treatment flasks: one flask with just spider silk chips, one with chips containing PGG at 500 μM, and one with chips containing CHG at 3.2 mg per chip.

These studies lasted 35 days. Cells in the flask with chips containing CHG died after the first day due to high CHG concentrations in the small flask volume. The remaining flasks grew normally, with no significant difference in growth or morphology between the control and treated cells. This experiment demonstrated the biocompatibility of spider silk and spider silk with a therapeutic.

It will be appreciated that variations of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A drug delivery composition, comprising:
a spider silk protein selected from MaSp1, MaSp2, MiSp1, MiSp2, tubuliform, flagelliform, piriform, aciniform, aggregate, and any combination thereof;
a medicinal agent; and
optionally a carrying agent.

2. The drug delivery composition of claim 1, wherein the medicinal agent is selected from: an antibiotic, an anti-inflammatory agent, a growth factor, an analgesic, a bioactive, and any combination thereof.

3. The drug delivery composition of claim 1, wherein the drug delivery composition is in a form selected from: a fiber, a solution, a gel, a hydrogel, a solid chip, a film, an adhesive, and a coating.

4. The drug delivery composition of claim 1, wherein the spider silk protein is a fiber.

5. The drug delivery composition of claim 1, further comprising a cross-linking agent.

6. A method of delivering a medicinal agent, comprising:
administering to a subject the drug delivery composition according to claim 1, wherein when the drug delivery composition is administered in a fluid or gel form, the composition fills voids on a substrate surface.

7. The method of claim 6, wherein the drug delivery composition is administered orally or dermally.

8. The method of claim 6, wherein the drug delivery composition is administered sub-gingivally.

9. The method of claim 6, further comprising curing the drug delivery composition before or after the administration step.

10. The method of claim 6, further comprising contacting a target area with the medicinal agent.

11. A method of preparing a drug delivery composition, comprising:
solubilizing a spider silk protein selected from MaSp1, MaSp2, MiSp1, MiSp2, tubuliform, flagelliform, piriform, aciniform, aggregate, and any combination thereof in an aqueous solution; and
adding a medicinal agent to the aqueous solution.

12. The method of claim 11, further comprising adding the drug delivery composition to a mold and allowing the drug delivery composition to solidify forming a solid chip in the form of the mold.

13. A method of treating periodontal disease, comprising:
administering to a subject the drug delivery composition prepared according to the method of claim 11.

14. An adhesive comprising the drug delivery composition of claim 1.

15. A coating on a medical device comprising the drug delivery composition of claim 1.
